(19) United States
(12) Patent Application Publication
(10) Pub. No.: US 2014/0288354 A1
(43) Pub. Date: Sep. 25, 2014

(54) FLUID TRANSPORT APPARATUS

(71) Applicants: Daniel Timms, Wilston (AU); Shaun David Gregory, Bracken Ridge (AU); Nicholas Richard Gaddum, Havelock North (NZ)

(72) Inventors: Daniel Timms, Wilston (AU); Shaun David Gregory, Bracken Ridge (AU); Nicholas Richard Gaddum, Havelock North (NZ)

(21) Appl. No.: 14/344,051

(22) PCT Filed: Sep. 7, 2012
(86) PCT No.: PCT/AU2012/001068

§ 371 Date: Mar. 10, 2014

(30) Foreign Application Priority Data
Sep. 9, 2011 (AU) 2011903674

(51) Int. Cl. A61M 1/10

(52) U.S. Cl. CPC A61M 1/1008
USPC 600/16

(57) ABSTRACT

Apparatus for transporting fluid within a biological subject, the apparatus including an at least partially compliant section, so that the compliant section at least partially controls the flow of fluid therethrough. The compliant section may also be used in conjunction with a heart pump for the purpose of at least partially controlling the flow of blood through the heart pump.

### BACKGROUND OF THE INVENTION

The present invention relates to an apparatus for transporting fluid within a biological subject, the apparatus including a compliant section for providing flow control. In one example the apparatus includes either a cannula for transporting fluid such as blood, or a heart pump for pumping blood.

### DESCRIPTION OF THE PRIOR ART

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

The use of mechanical device therapy to treat heart failure is increasing as the general population ages and the number of donor organs for heart transplantation remains limited. Devices can be used to bridge a patient to heart transplant, to recovery, or indeed as a destination alternative. The latter support strategy requires a device with increased mechanical durability/lifetime.

Mechanical durability is dependent on the functionality of the device, in particular, the type of bearings implemented. First generation pulsatile devices necessitate contacting components, which limits their predicted mechanical lifetime below three years. The reduced size of second generation non-pulsatile rotary impeller devices has accelerated them to the forefront of VAD (ventricular assist device) development.

However, initial techniques for impeller support also imposed significant limitations on device lifetime, as they required a shaft, seals and bearings U.S. Pat. No. 4,589,822. Subsequent improvements resulted in devices that rely on blood immersed pivot support U.S. Pat. No. 5,601,418; however, predicted service life is still below five years.

Several techniques have since been developed to improve device lifetime, ranging from complete magnetic suspension U.S. Pat. No. 6,575,717, to passive hydrodynamic suspension U.S. Pat. No. 6,227,797. These third generation devices eliminate contact wear and reduce the number of moving components, potentially increasing lifetime to beyond ten years. These latest generation suspension techniques eliminate any point to point contact which may also improve the hemolytic performance of the pump.

The control of blood flow through a heart pump is critical to ensure successful operation of the pump, and in particular to meet a patient's blood flow requirements. For example, the ability to maintain a balance between the outflow of a LVAD (left ventricular assist device) and the natural right heart, the left and right outflow of a BiVAD (Bi-Ventricular Assist Device), and a TAH (Total Artificial Heart) system, is important for successful device operation. Haemodynamic parameters that may upset this balance include the bronchial flow, relative changes in systemic and pulmonary vascular resistance, relative changes in left and right ventricular contractility, pulmonic or systemic congestion, and ventricular collapse. These conditions infer that a technique for balancing the left and right VAD hydraulic output is required for long term support.

Previous attempts to balance the left/right outflow requirements in a BiVAD have often relied on the use of a pressure sensor to detect left atrial pressure (LAP). A feedback mechanism is then employed to either reduce LVAD speed, or increase RVAD speed, in the presence of reduced LAP. Another technique includes the surgical introduction of a shunt between the left and right atrium to safely protect against the potentially disastrous build up of fluid in either atrium.

Alternatively, U.S. Pat. No. 6,527,698 includes a conduit linking right to left atria through which flow is varied via a variable occluding valve. However, this technique introduces an additional blood contacting conduit, as well as complexities involved with active feedback control, such as the need for sensors. Furthermore, this solution can help to balance the fluid distribution but does not provide a method for controlling the alteration of device outflow.

WO2006053384A1 describes axially displacing a rotating dual impeller within a cavity so as to simultaneously alter the relative efficiencies of each side of the device. However, this application describes the control method used to achieve this axial displacement as active, thus requiring the use of feedback signals from pressure sensors and the like to actively control and maintain a desired set axial location. This method of control may inherently consume excessive amounts of electrical power.

WO2010118476 describes a controller for a heart pump, the controller including a processing system for determining movement of an impeller within a cavity in a first axial direction, the cavity including at least one inlet and at least one outlet, and the impeller including vanes for urging fluid from the inlet to the outlet, causing a magnetic bearing to move the impeller in a second axial direction opposite the first axial direction, the magnetic bearing including at least one coil for controlling an axial position of the impeller within the cavity, determining an indicator indicative of the power used by the magnetic bearing and causing the magnetic bearing to control the axial position of the impeller in accordance with the indicator to thereby control a fluid flow between the inlet and the outlet.

All of the above techniques control the hydraulic output from the heart pump by controlling parameters such as motor power, speed, and axial position of the impeller, using information such as the flow pressure, pump speed and the like.

Furthermore, whilst determining the motor power and speed is relatively easy, detecting the remaining parameters conventionally requires additional instrumentation, such as pressure sensors and flow meters. These components increase the possibility of device failure; as such components have limited long term reliability. Furthermore, their addition to the device can induce extra blood contact with other foreign material, exacerbating the potential for blood damage.

A further issue is that active control systems reliant on sensors are prone to drift or use inferred data which may be inaccurate under certain situations such as partial/full inflow occlusion. This in turn can render the pump ineffective in some scenarios.

U.S. Pat. No. 6,790,171 describes a flexible, collapsible cannula used for easy and non-invasive attachment of a VAD. The flexible cannula can be guided into the required vessel through a less invasive site, rather than requiring invasive implantation. The collapsible nature of the cannula allowed for a smaller incision and easier implantation.

US 2011/0118537 describes a collapsible inflow cannula which can reduce in size to allow for easier and less invasive implantation.

U.S. Pat. No. 6,312,443 describes an expandable cannula which can have two shapes only, the expanded shape and the collapsed shape. The collapsed shape is used for ease of implantation and for a minimally invasive procedure, while the expanded shape is used during operation to allow a larger flow passage.

US2003/0023131 describes a technique of designing the cannulae of specific dimensions and mechanical characteristics (eg. Compliance) to provide a required inertia to prevent backflow through the device in the event of device malfunction.

### SUMMARY OF THE PRESENT INVENTION

The present invention seeks to ameliorate one or more of the problems associated with the prior art.

In a first broad form the present invention relates to apparatus for transporting fluid within a biological subject, the apparatus including an at least partially compliant section, so that the compliant section at least partially controls the flow of fluid through the apparatus.

Typically a cross sectional area of at least part of the compliant section is dependent at least partially on the pressure of fluid therein, the cross sectional area of the compliant section controlling the flow of fluid therethrough.

Typically the cross sectional area of at least part of the compliant section is dependent on a pressure gradient across a wall of the compliant section.

Typically compliance is provided by at least one of:
a) a resilient band;
b) a compressible fluid; and,
c) material properties of the compliant section.

Typically the compliant section includes a deformable tube.

Typically the compliant section includes a resilient band provided around at least part of the deformable tube.

Typically the compliant section at least one of
a) includes a rigid external casing;
b) is mounted within a rigid external casing;
c) includes a rigid external support;
d) includes a semi-rigid external wire reinforcement.

Typically the compliant section includes:
a) a substantially rigid outer casing;
b) a deformable tube provided within the casing; and,
c) a compressible fluid provided between the deformable tube and the casing.

Typically at least part of the compliant section is made of at least one of:
a) silicone;
b) a woven fibre material; and,
c) silastomer P15.

Typically in an expanded state at least part of the compliant section has an inner diameter of at least one of
a) between 1 mm and 25 mm
b) between 5 mm and 15 mm; and,
c) about 12 mm.

Typically in a restricted state at least part of the compliant section has an inner diameter of at least one of
a) between 1 mm and 10 mm;
b) between 2 mm and 8 mm; and,
c) about 6 mm.

Typically the compliant section has a wall thickness of at least one of
a) between 0.5 mm and 3 mm; and,
b) about 1 mm.

Typically the compliant section has a compliance of at least one of
a) between 0.2 mL/mmHg and 0.01 mL/mmHg;
b) between 0.1 mL/mmHg and 0.05 mL/mmHg;
c) about 0.08 mL/mmHg;
d) between 0.02 mL/mmHg and 0.001 mL/mmHg;
e) between 0.01 mL/mmHg and 0.005 mL/mmHg; and,
f) about 0.007 mL/mmHg.

Typically the apparatus includes a cannula.

Typically the cannula is attached to at least one of an inlet and an outlet of a heart pump.

Typically the apparatus includes a heart pump, the compliant section being associated with at least one of an inlet and an outlet of the heart pump.

Typically at least part of an inlet compliant section moves between an expanded state under conditions of normal inlet pressure and a restricted state under conditions of reduced inlet pressure, thereby reducing blood flow through the heart pump as the inlet pressure decreases.

Typically at least part of an outlet compliant section moves between a restricted state under conditions of normal outlet pressure and an expanded state under conditions of increased outlet pressure, thereby increasing blood flow through the pump as the outlet pressure increases.

In a second broad form the present invention relates to a heart pump for pumping blood, the heart pump including:
a) a cavity including at least one inlet and at least one outlet;
b) an impeller provided within the cavity, the impeller including vanes for urging blood from the inlet to the outlet; and,
c) a drive for rotating the impeller in the cavity; and,
d) an at least partially compliant section associated with at least one of the inlet and the outlet, wherein in use the compliant section at least partially controls the flow of blood through the heart pump.

Typically a cross sectional area of at least part of the compliant section is at least partially dependent on the pressure of blood therein, the cross sectional area of the compliant section controlling the flow of blood through the heart pump.

Typically the cross sectional area of at least part of the compliant section is dependent on a pressure gradient across a wall of the compliant section.

Typically at least part of an inlet compliant section moves between an expanded state under conditions of normal inlet pressure and a restricted state under conditions of reduced inlet pressure, thereby reducing blood flow through the pump as the inlet pressure decreases.

Typically at least part of an outlet compliant section moves between a restricted state under conditions of normal outlet pressure and an expanded state under conditions of increased outlet pressure, thereby increasing blood flow through the pump as the outlet pressure increases.

Typically the compliant section is part of at least one of:
a) the inlet;
b) the outlet;
c) a volute associated with the outlet;
d) an inlet cannula coupled to the inlet for receiving blood from a subject; and,
e) an outlet cannula coupled to the outlet for supplying blood to the subject,

Typically compliance is provided by at least one of:
a) a resilient band;
b) a compressible fluid; and,
c) material properties of the compliant section.

Typically the compliant section includes a deformable tube.

Typically the compliant section includes a resilient band provided around at least part of the deformable tube.

Typically the compliant section at least one of:
a) a rigid external casing;
b) is mounted within a rigid external casing;
c) a rigid external support;
d) includes a semi-rigid external wire reinforcement.

Typically the compliant section includes:
a) a substantially rigid outer casing;
b) a deformable tube provided within the casing; and,
c) a compressible fluid provided between the deformable tube and the casing.

Typically at least part of the compliant section is made of at least one of:
a) silicone;
b) a woven fibre material; and,
c) silastomer P15.

Typically in an expanded state at least part of the compliant section has an inner diameter of at least one of:
a) between 1 mm and 25 mm;
b) between 5 mm and 15 mm; and,
c) about 12 mm.

Typically in a restricted state at least part of the compliant section has an inner diameter of at least one of:
a) between 19 mm and 190 mm;
b) between 2 mm and 8 mm; and,
c) about 6 mm.

Typically the compliant section has a wall thickness of at least one of:
a) between 0.5 mm and 3 mm; and,
b) about 1 mm.

Typically the compliant section has a compliance of at least one of:
a) between 0.2 mL/mmHg and 0.01 mL/mmHg;
b) between 0.1 mL/mmHg and 0.05 mL/mmHg; 4
c) about 0.08 mL/mmHg;
d) between 0.02 mL/mmHg and 0.001 mL/mmHg;
e) between 0.01 mL/mmHg and 0.005 mL/mmHg; and,
f) about 0.007 mL/mmHg.

Typically the drive includes:
a) a first magnetic material provided in the impeller;
b) at least one drive coil that in use generates a magnetic field that cooperates with the first magnetic material allowing the impeller to be rotated.

Typically the first magnetic material includes a number of circumferentially spaced permanent magnets mounted in the impeller, adjacent magnets having opposing polarities.

Typically the pump includes a magnetic bearing including at least one bearing coil for maintaining an axial position of the impeller.

Typically, in use, the at least one bearing coil generates a magnetic field that cooperates with second magnetic material in the impeller, allowing the axial position of the impeller to be maintained.

Typically the second magnetic material is a permanent magnet.

Typically the at least one bearing coil is for generating a magnetic field that is one of complementary to and counter to the first magnetic field generated by the permanent magnet, thereby controlling the net magnetic field between the bearing and the first magnetic material.

Typically the heart pump includes:
a) a first cavity portion having a first inlet and a first outlet;
b) a second cavity portion having a second inlet and a second outlet; and,
c) first and second sets of vanes provided on the impeller, each set of vanes being for urging fluid from a respective inlet to a respective outlet.

Typically the pump includes a connecting tube extending between the first and second cavity portions, the impeller including:
a) a first set of vanes mounted on a first rotor in the first cavity portions;
b) a second set of vanes mounted on a second rotor in the second cavity portion; and,
c) a shaft connecting the first and second rotors, the shaft extending through the connecting tube.

Typically the at least one of the drive and a magnetic bearing are provided at least partially between the first and second cavity portions

Typically first magnetic material is mounted in a first rotor supporting the first set of vanes and wherein the magnetic bearing is positioned adjacent the first cavity, the magnetic bearing and first magnetic material being configured to result in an attractive force between the magnetic bearing and the first rotor.

Typically second magnetic material is mounted in a second rotor supporting the second set of vanes and wherein the magnetic bearing is positioned adjacent the second cavity, the magnetic bearing and second magnetic material being configured to result in an attractive force between the magnetic bearing and the second rotor.

In a third broad form the present invention relates to a cannula for transporting fluid within a biological subject, the cannula including an at least partially compliant section, so that the compliant section at least partially controls the flow of fluid through the cannula.

Typically a cross sectional area of at least part of the compliant section is dependent at least partially on the pressure of fluid therein, the cross sectional area of the compliant section controlling the flow of fluid therethrough.

Typically the cross sectional area of at least part of the compliant section is dependent on a pressure gradient across a wall of the compliant section.

Typically compliance is provided by at least one of:
a) a resilient band;
b) a compressible fluid; and,
c) material properties of the compliant section.

Typically the compliant section includes a deformable tube.

Typically the compliant section includes a resilient band provided around at least part of the deformable tube.

Typically the compliant section at least one of:
a) includes a rigid external casing;
b) is mounted within a rigid external casing;
c) includes a rigid external support;
d) includes a semi-rigid external wire reinforcement.

Typically the compliant section includes:
a) a substantially rigid outer casing;
b) a deformable tube provided within the casing; and,
c) a compressible fluid provided between the deformable tube and the casing.

Typically at least part of the compliant section is made of at least one of:
a) silicone;
b) a woven fibre material; and,
c) silastomer P15.

Typically in an expanded state at least part of the compliant section has an inner diameter of at least one of:
a) between 1 mm and 25 mm;
b) between 5 mm and 15 mm; and,
c) about 12 mm.

Typically in a restricted state at least part of the compliant section has an inner diameter of at least one of
a) between 1 mm and 10 mm;
b) between 2 mm and 8 mm; and,
c) about 6 mm.

Typically the compliant section has a wall thickness of at least one of:
a) between 0.5 mm and 3 mm; and,
b) about 1 mm.

Typically the compliant section has a compliance of at least one of
a) between 0.2 mL/mmHg and 0.01 mL/mmHg;
b) between 0.1 mL/mmHg and 0.05 mL/mmHg;
c) about 0.08 mL/mmHg;
d) between 0.02 mL/mmHg and 0.001 mL/mmHg;
e) between 0.01 mL/mmHg and 0.005 mL/mmHg; and,
f) about 0.007 mL/mmHg.

### BRIEF DESCRIPTION OF THE DRAWINGS

An example of the present invention will now be described with reference to the accompanying drawings, in which:—

FIG. 1A is a schematic cross sectional view of a first example of a heart pump;

FIG. 1B is a schematic cross sectional view of a second example of a heart pump;

FIGS. 2A to 2C are schematic side views of an example of an inlet cannula under conditions of different inlet pressures;

FIGS. 2D to 2F are schematic side views of an example of an outlet cannula in under conditions of different outlet pressures;

FIGS. 2G and 2H are schematic cross sectional views of example cannulae;

FIGS. 3A to 3C are graphs showing example flow parameters for two rotary pumps having compliant and rigid inlet cannulae, respectively;

FIGS. 4A to 4H are graphs showing further example flow parameters for two rotary pumps having compliant and rigid inlet cannulae, respectively;

FIG. 5A shows graphs of example left atrial pressure (LAP), inflow cannula resistance (LVADRin) and flow rate (LVADQ) for atrial compliant and rigid inflow cannulae during severe left heart failure, before, during and after an increase in pulmonary vascular resistance;

FIG. 5B shows graphs of example left ventricular volume (LVV), inflow cannula resistance (LVADRin) and flow rate (LVADQ) for compliant ventricular inflow cannulae during severe left heart failure, before, during and after an increase in pulmonary vascular resistance;

FIG. 5C shows graphs of example left ventricular volume (LVV), inflow cannula resistance (LVADRin) and flow rate (LVADQ) for rigid ventricular inflow cannulae during severe left heart failure, before, during and after an increase in pulmonary vascular resistance;

FIG. 6A shows graphs of example left ventricular volume (LVvol) and right ventricular volume (RVvol) after a change in pulmonary vascular resistance (PVR) for rigid outflow cannulae;

FIG. 6B shows graphs of example left ventricular volume (LV Volume) after a gradual change in pulmonary vascular resistance (PVR) for compliant outflow cannulae;

FIG. 7A is a schematic diagram of a further example of a compliant section of a cannula in a restricted configuration;

FIG. 7B is a schematic diagram of the cannula of FIG. 7A in a partially expanded configuration;

FIG. 7C is a schematic diagram of the cannula of FIG. 7A in a fully expanded configuration;

FIG. 7D is a schematic diagram of the cannula of FIG. 7A showing example dimensions;

FIG. 8A is a schematic cross sectional view of a third example of a heart pump; and,

FIG. 8B is a schematic perspective view of the impeller of the heart pump of FIG. 8A.

### DETAILED DESCRIPTION OF THE PREFERRED EMBODIMENTS

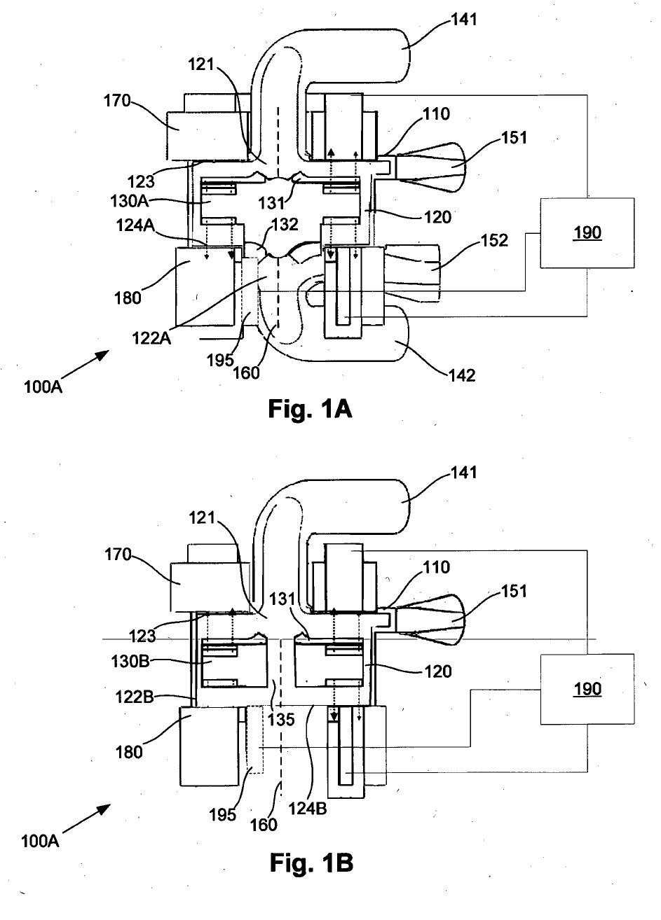

A first example of a heart pump will now be described with reference to FIG. 1A.

In this example, the heart pump 100A includes a housing 110 defining a cavity 120, containing an impeller 130A. The impeller 130A effectively divides the cavity 120 into first and second cavity portions 121, 122A. The housing 110 includes first and second inlets 141, 142 and corresponding first and second outlets 151, 152, which are in fluid communication with the first and second cavity portions 121, 122A, respectively. It will be appreciated that the cavity 120 may include volutes (not shown) to assist with transfer of the fluid to the outlets 151, 152.

The impeller 130A includes first and second sets of vanes 131, 132, such that rotation of the impeller 130A about a rotation axis 160 urges fluid from the inlets 141, 142 to the corresponding outlets 151, 152. In use, rotation of the impeller 130A is achieved using a drive, such as a magnetic drive 170. The magnetic drive 170 typically includes at least one coil positioned at a first end of the housing 110 adjacent the first cavity 121. In use, the coil generates a magnetic field that cooperates with magnetic material in the impeller 130A, allowing the impeller to be rotated.

A magnetic bearing 180 may also be provided including at least one coil positioned at a second end of the housing 110 adjacent the second cavity 122A. In use, the coil generates a magnetic field that cooperates with magnetic material also in the magnetic bearing stator, which interacts with ferrous material within the impeller 130A. This generates an attractive force between the magnetic bearing 180 and the impeller 130A, to balance attractive forces generated by the drive 170 and thereby levitate the impeller.

In use as a BiVAD, the heart pump 100A is arranged so that the pumping action provided by each set of vanes 131, 132 equates to the pumping action required by each of the left and right ventricles respectively. This can be achieved by selection of suitable dimensions, such as the length, height and shape of the respective vanes, as well as an impeller speed and vane clearance.

In one example, with the impeller 130A operating at a set rotational speed of approximately 2300 rpm, this results in the desired haemodynamics of 100 mmHg (LVAD) and 20 mmHg (RVAD) being produced for the systemic and pulmonary systems. Accordingly, the flows via the first and second outlets 151, 152 are in balance at approximately 5 L/min. The exact rate of flow from the left cavity is slightly higher than the right cavity, due to the natural outflow differential of the heart caused by the bronchial circulation.

Depending on impeller geometry, this normal pressure differential may lead to a force on the impeller 130A, for example acting towards the second cavity portion 122A. However, the heart pump is typically naturally balanced, so that any such forces on the impeller 130A including forces resulting from the pressure differential and the attractive forces caused by magnetic coupling between the impeller 130A and the drive 170, as well as between the impeller 130A and the bearing 180, are approximately equal, thereby maintaining the impeller position. This minimises the electrical power used by the bearing 180.

It will be appreciated that the drive 170 and magnetic bearing 180 are typically coupled to a controller 190, thereby allowing the drive and axial bearing to be operated. In one example, this is performed to maintain a constant impeller position and rotational speed, which can be achieved using simple position/movement sensors, thereby avoiding the need for complex flow and pressure sensors. This also allows for a simple control algorithm, thereby making the pump easier to manufacture and operate, and less prone to error.

A second example of a heart pump will now be described with reference to FIG. 1B. In this example, similar reference numerals are used to designate similar features, and these will not therefore be described in any detail.

In this example, the heart pump 100B includes a modified second cavity 112B, having a surface 124B that extends across the housing 110, where the inlet is provided in the example of FIG. 1A. Accordingly, in this example, the heart pump 110 does not include a second inlet or a second outlet. Furthermore, impeller 130B includes only a single set of vanes 131, positioned in the cavity 121, and includes an aperture 135 extending through the impeller 130B, for allowing blood to flow from the second cavity 122B to the first cavity 121, to thereby prevent stagnation between the impeller 130B and the second cavity surface 124B.

Otherwise the heart pump 100B is substantially the same as the heart pump 100A, and will not therefore be described in further detail.

As will be come apparent from the description below, the above described heart pumps, and in particular the use of a magnetically suspended and/or driven impeller is for the purpose of example, and alternative arrangements can be used.

In any event, in use, the heart pumps 100A, 100B can be connected to a subject using cannula connected to the inlets and/or outlets, or by connecting the inlet and/or outlet directly to the heart. This allows the heart pumps 100A, 100B to supplement the pumping action of one or both of the left and right ventricles of the heart.

For example, the heart pump 100A of FIG. 1A can be coupled to both the pulmonary and systemic circulatory systems, allowing the pump to operate as a BiVAD (Bi-Ventricular Assist Device), in which the pump supplements the pumping action of both the left and right ventricles of the heart. In this instance, the left ventricle and the right atrium are coupled to the first and second inlets 141, 142 respectively, whilst the first and second outlets 151, 152 and provide outflow to the aorta and the pulmonary artery, respectively.

In use, the inlet or outlet is associated with a compliant section that is at least partially compliant to thereby control the flow of blood through the heart pump. In particular, the cross sectional area of at least part of the compliant section is dependent at least partially on the pressure of blood therein, so that as the blood pressure changes, so does the cross sectional area of the compliant section, and hence the rate of blood through the corresponding inlet or outlet. This in turn adjusts the flow of blood through the heart pump, allowing the pressure change to be compensated for.

The cross sectional area of the compliant section may also be dependent on a pressure gradient across a wall of the compliant section, and can for example depend on an external pressure surrounding the compliant section which may be actively or passively changed. For example, if the pressure of an external gas or fluid about the compliant section is actively or passively changed, the cross sectional area of the compliant section will also change, and hence change the rate of blood through the corresponding inlet or outlet.

The use of a compliant section can therefore allow the heart pump to passively accommodate changes in blood flow within the circulatory system, without requiring the need for active control of the impeller. Accordingly, this allows the impeller position and/or rotational speed to be maintained constant, whilst the flow of blood through the pump is at least partially passively adjusted by the compliant section. This can be used to eliminate the need for complex sensor or sensor-less based control systems, and therefore reduce associated problems, such as sensor drift (sensor based), inaccurate inferred data (sensor-less based), controller malfunction (sensor and sensor-less based).

However, it will be appreciated that the above described compliant section could be used in conjunction with other flow control techniques, such as through the use of a zero power controller described for example in copending applications WO2010/118475 and WO2010/118476.

A number of further features will now be described.

The compliant section could form part of an inlet cannula coupled to the inlet for receiving blood from a subject, or part of an outlet cannula coupled to the outlet for supplying blood to the subject. Compliance of a cannula can be achieved either by making the entire cannula compliant, or by including a compliant section. The term compliant cannula is therefore interpreted to mean a cannula with a compliant section, as well as a cannula that is compliant along the entire cannula length.

However, the compliant section may alternatively form part of the inlet or outlet, or a volute associated with the outlet. Whilst the remainder of the description will focus on the use of compliant cannulae it will be appreciated that this is not intended to be limiting and similar principles will apply to compliant sections provided as part of the inlet, outlet or outlet volute.

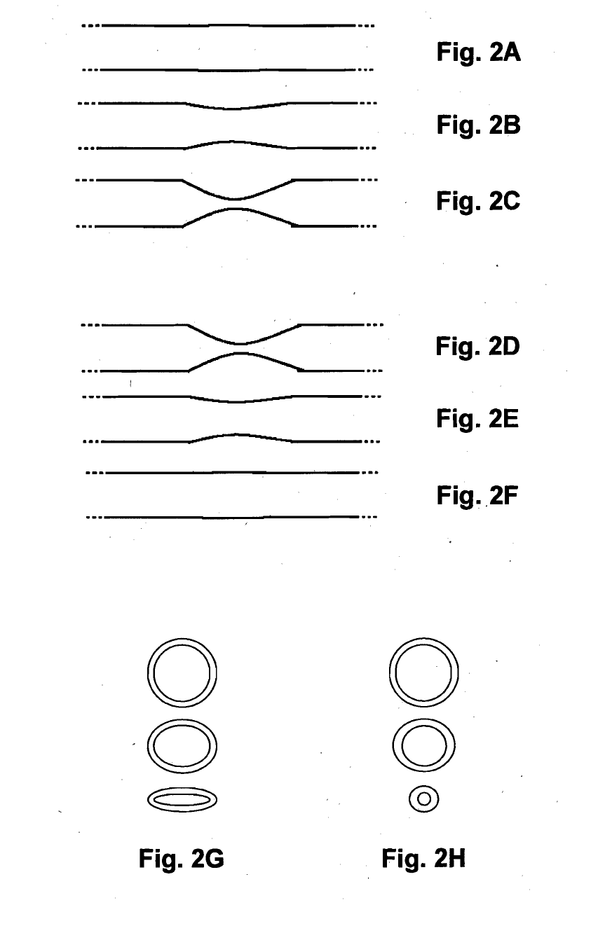

When associated with an inlet the compliant section can move between an expanded state under conditions of normal inlet pressure and a restricted state under conditions of reduced inlet pressure, thereby reducing blood flow through the pump as the inlet pressure decreases, as will now be described with reference to FIG. 2A to 2C.

In this example, under normal conditions, the cannula 200 is in an expanded state, as shown in FIG. 2A. As pressure in the inlet cannula drops, a part of the cannula 201 progressively restricts as a result of the compliance of the cannula, as shown in FIGS. 2B and 2C. Accordingly, as the pressure of blood in the inlet cannula drops, the cannula restricts so as to restrict the flow of blood along the cannula, thereby increasing the resistance of the VAD circuit and preventing collapse of the inlet vessel/chamber. This reduction in flow serves to control VAD output flow based on preload (inlet pressure) and therefore increases the preload sensitivity of rotary heart pumps.

Restriction of the inflow cannula is dependent on the pressure differential across the compliant wall, so assuming a constant external pressure in an implanted or extracorporeal situation, the restriction is due entirely to the pressure within the compliant section of the cannula. If placed close to the cannulation site, the restriction is primarily due to the pressure within the cannulated blood vessel. Therefore, as the pressure in the cannulated vessel reduces, the compliant segment begins to restrict and passively control the cardiac output (VAD flow rate) dependant on preload (cannulated vessel pressure).

The compliant inflow cannula can be used as a passive flow modulator in LVAD, RVAD and BiVAD scenarios (dual LVAD, single impeller LVAD/BiVAD), with the operation being substantially the same as the compliant segment is only required to collapse under low pressures in the cannulated chamber (left, or right atrium or ventricle).

Conversely when associated with an outlet, the compliant section moves between a restricted state under conditions of normal outlet pressure and an expanded state under conditions of increased outlet pressure, thereby increasing blood flow through the pump as the outlet pressure increases, as will now be described with reference to FIG. 2D to 2F.

In this example, at least part of the outlet cannula 211 moves between a restricted state under conditions of normal outlet pressure, as shown in FIG. 2D, progressively expanding as the outlet pressure increases, as shown in FIGS. 2E and 2F. Thus, in an unstressed state, the cannula has an hour glass shape. As pressure of blood in the outlet cannula increases, the cannula expands, decreasing the resistance of the VAD circuit, and hence increasing the VAD outlet flow, whilst a low afterload will sufficiently reduce the compliant segment diameter to control the flow rate. This increase in outlet flow due to increased outlet pressure decreases the afterload (outlet pressure) sensitivity of a rotary heart pump for use as left and/or right VAD support.

Thus, the incorporation of a compliant section in an outlet cannula of the right pump and/or left pump of a biventricular assist or total artificial heart system (dual or single device) will further assist in providing a self-regulating/balancing mechanism for the outflow of both left and right pumps, which are connected in series. This will assist in reducing the likelihood of the collapse of either heart chamber, which may occur due to the rapid evacuation of fluid from one chamber, which cannot be replaced in a timely fashion by the other pump.

However, it will be appreciated that similar results are achieved if the compliant section forms part of the inlet or outlet.

Accordingly, a compliant section associated with the inlet can be used to increase preload sensitivity of rotary heart pumps operating as a VAD. This therefore helps reduce and even eliminate suction events caused by the lack of preload sensitivity in rotary VADs, which in turn helps prevent the associate harmful consequences such as endocardial damage, ventricular arrhythmias, pump flow stoppages, or the like.

Similarly, a compliant section associated with the outlet can be used to decrease afterload sensitivity of rotary heart pumps. Such, a decrease in afterload sensitivity of a rotary VAD allows the VAD to passively respond to changes in outlet pressure caused by events such as systemic or pulmonary vascular resistance changes.

The compliant section may include a compliant and/or deformable tube, for example if this is provided as part of a cannula. In this case, additional structural support may be required so that the compliant section is not deformed by external forces, such as compression of the compliant section by internal organs within the subject. In one example, the compliant section can be mounted within, or include, a rigid casing. In another example, the compliant section includes a rigid external support or semi-rigid external wire reinforcement. It will be appreciated that other structural support arrangements could also be used, if required, such as a structural support surrounding the compliant section, but not necessarily attached thereto, to stent open the space around the cannula and allow expansion of the compliant section, as required.

The compliance allows the shape of the compliant section to adjust under conditions of varying pressure and it will therefore be appreciated that the term compliance should be understood to include any arrangement that allows this functionality to be achieved.

In one example, the compliance can arise as a result of the material properties from which the cannula is constructed. For example, the cannula can be made of a suitable material that includes a degree of inherent compliance, such as silicone materials, like Silastomer P15. The materials are also typically biologically inert.

Additionally, and/or alternatively, if the material from which the compliant section is formed does not provide sufficient resilience, a mechanism can be provided that introduces resilience, so that the tube returns to a normal shape under normal pressures. In one example, resilience can be caused by a resilient band provided around at least part of a deformable tube to thereby provide the compliance. Thus, a band of compliant or elasticised material can be provided circumferentially around the cannula to ensure desired properties are achieved.

Alternatively, resilience can arise from the use of a compressible fluid, such as an inert gas, provided on an outside of a deformable tube, for example between the tube and an outer casing, as will be described in more detail below.

Similarly compliance of the inlet or outlet can be achieved using any suitable technique, such as manufacturing part of the inlet or outlet from a compliant material, or mounting a compliant section within the inlet or outlet. This will not therefore be described in further detail.

The dimensions of the compliant sections will be selected based on the intended flow rates, the degree of compliance required, and the particular pump configuration being used.

For example, for an inlet cannula, in an expanded state, the compliant section has an inner diameter of between 1 mm and 20 mm, more typically between 5 mm and 15 mm and generally about 12 mm. In a restricted state, by contrast, the compliant section has a typical inner diameter of between 1 mm and 10 mm, between 2 mm and 8 mm and more typically about 6 mm. The compliant section wall thickness is typically between 0.5 mm and 3 mm and more typically is about 1 mm. Thus, in one example, for an inlet cannula, the compliant section has a compliance of between 0.2 mL/mmHg and 0.01 mL/mmHg, typically between 0.1 mL/mmHg and 0.05 mL/mmHg and more typically about 0.08 mL/mmHg.

In contrast for an outlet cannula, the compliant section has a compliance of between 0.02 mL/mmHg and 0.001 mL/mmHg, typically between 0.01 mL/mmHg and 0.005 mL/mmHg and more typically about 0.007 mL/mmHg. Example outlet cannula diameters are 4 mm, 5 mm and 6 mm for mean pulmonary arterial pressures (mPAP) of 3 mmHg, 36 mmHg and 71 mmHg, respectively. Accordingly, the outlet cannula typically increases in diameter by approximately 1 mm for every 35 mmHg outflow pressure change, thereby maintaining a 5 L/min flow.

It will be appreciated that these values are however for the purpose of example only, and that in practice properties of the compliant sections, including the dimensions and compliance, will depend on a range of factors such as the operating parameters of the particular heart pump used, the recipient's condition, or the like. Thus, the examples provided for inlet cannula may also apply to outlet cannula, and vice versa.

Thus, for example, for an LVAD operating as an LVAD with passive flow control the ranges of the compliant section are also 5-25 mm, with the typical ‘restriction’ being about 12 mm, depending on the particular configuration used.

Example cross sectional shapes of the compliant sections in a variety of restricted and expanded states are shown in FIGS. 2G and 2H. It will be appreciated from this that a variety of different cross sectional shapes can be used, and that compliant sections do not need to restrict symmetrically.

Results of experimental operation of a heart pump including a compliant cannula will now be described. This study was completed in-vitro using a mock circulation loop. The mock circulation loop was a 5 element Windkessel model with systemic and pulmonary circulations. The heart consists of passively filling and Starling responsive ventricles. The ventricles exhibit a Starling response by automatically adjusting based on preload. Cannulation can be achieved in the left or right atria or ventricles for inflow and the ascending aorta and pulmonary artery for outflow.

In the experiment, a compliant segment was added to the inflow cannula of an LVAD and RVAD. In this regard, a thin walled, compliant inflow cannula was made with silicone of shore hardness 15 (Silastomer P15) by coating a 12 mm plastic rod. The wall thickness of the compliant segment was 2 mm, resulting in a compliance of 0.08 mL/mmHg. Following removal of the rod, the compliant cannula was attached as part of the inflow cannula for a two heart pump configuration in a mock circulation loop.

Pump speed was incrementally increased (100 RPM each time) until a maximum speed of 3000 RPM was reached. Both atrial and ventricular cannulation were evaluated for LVAD and RVAD support, under various degrees of VAD preload, VAD speed and systemic and pulmonary resistance. The experiment was also repeated for a rigid inflow cannula for comparison.

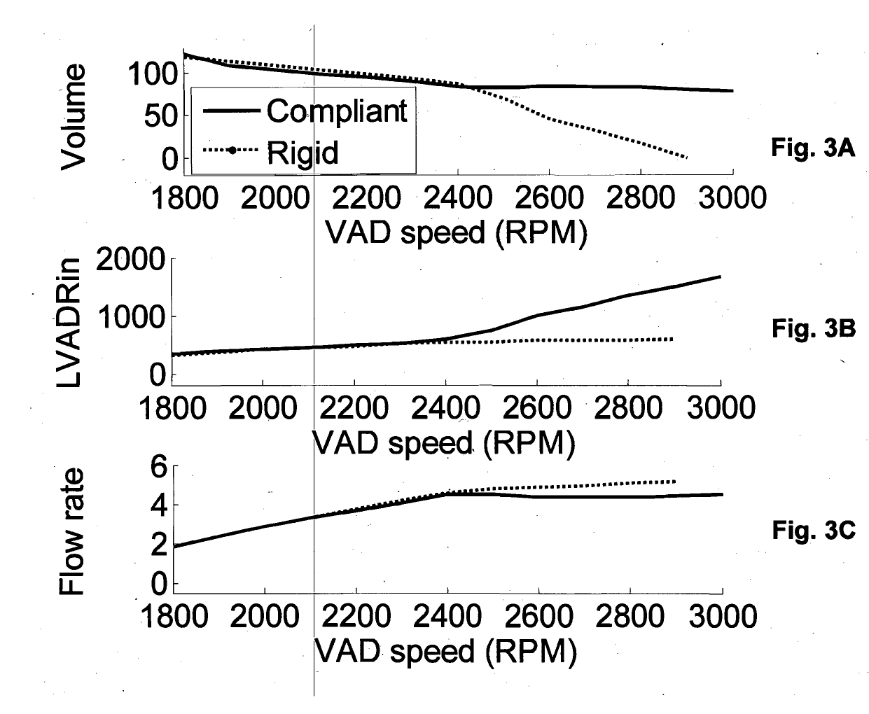

FIGS. 3A to 3C show results for an LVAD in left ventricular cannulation with both the compliant and semi-rigid inflow cannulae. FIGS. 3A to 3C represent the left ventricular volume (ml), the LVAD inflow cannula resistance (mmHg·s·cm⁻²) and the systemic flow rate (L/min), respectively.

As the LVAD speed was gradually increased, the ventricular volume was reduced. This trend continued with the rigid cannula as the inflow cannula resistance was relatively constant and the flow rate gradually increased. As the VAD speed reached 2900 RPM, left ventricular volume had reduced to zero and a suction event occurred. However, with the compliant inflow cannula, the LVAD inflow cannula resistance began to increase due to partial restriction of the compliant segment at LVAD speeds of approximately 2400 RPM. This passively reduced the LVAD flow rate (and hence systemic flow rate) and maintained positive left ventricular volumes, thereby preventing suck down.

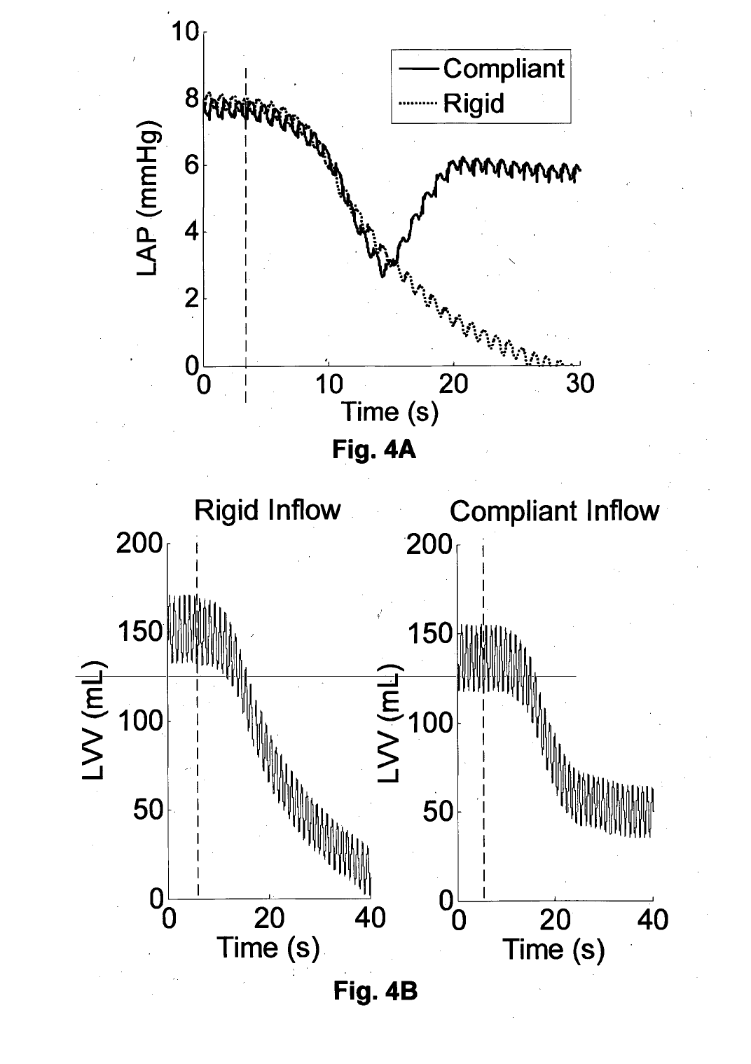

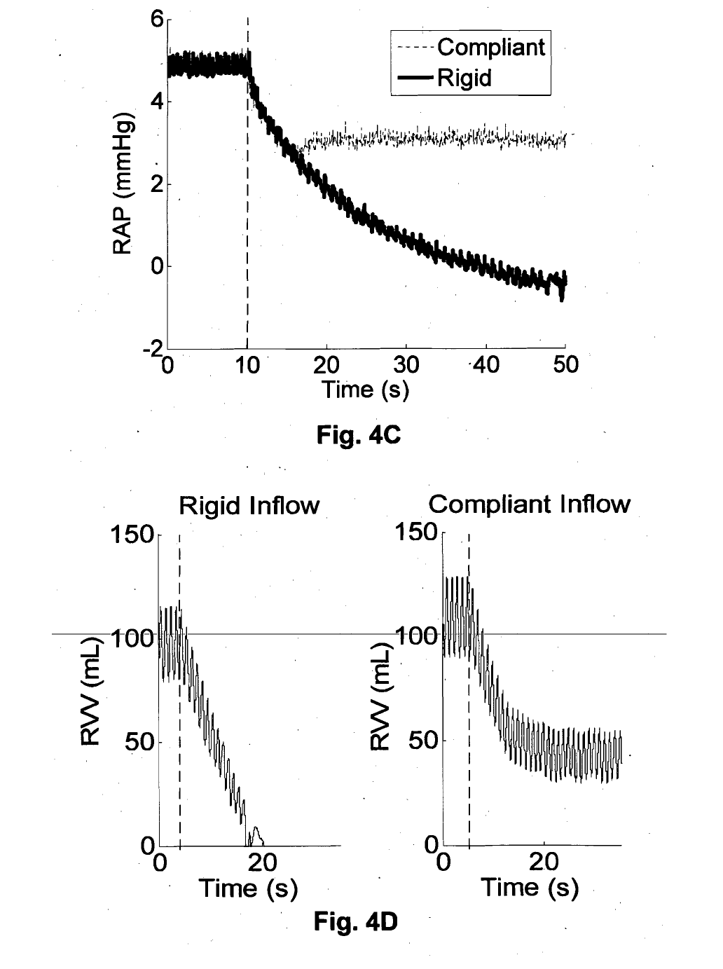

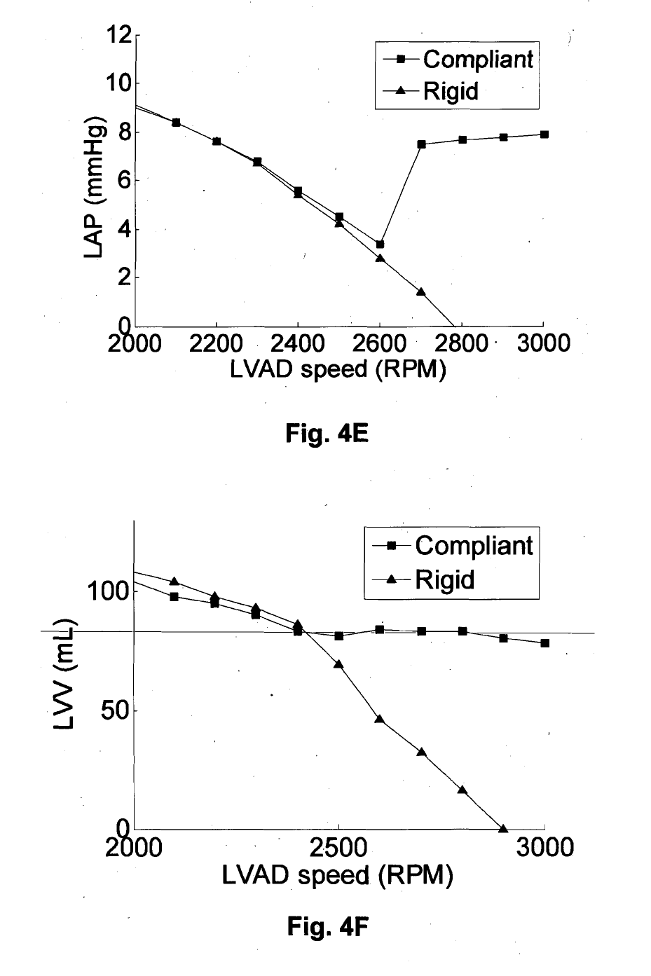

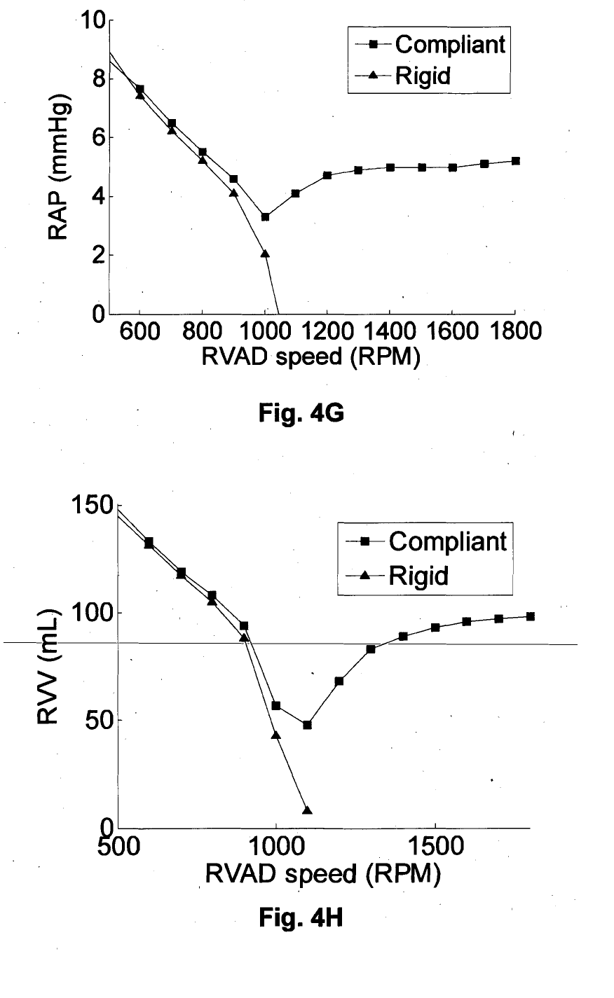

Further examples will now be described with reference to FIGS. 4A to 4H.

In FIGS. 4A and 4B, left atrial cannulation (described by changes in LAP) and left ventricular cannulation (described by changes in Systolic Left Ventricular Volume) respectively, are shown for an LVAD. In this example, the pulmonary vascular resistance (PVR) is increased from 100 to 400 dynes·s·cm⁻⁵ at the time point indicated in dotted lines, to thereby reduce LVAD preload. As shown, the compliant cannula is able to counteract the reduced LVAD preload, thereby maintaining flow, pressure and volume as compared to the rigid cannula.

In FIGS. 4C and 4D, right atrial cannulation (described by changes in RAP) and right ventricular cannulation (described by changes in Systolic Right Ventricular Volume) respectively, are shown for an RVAD. In this example, systemic vascular resistance (SVR) increased from 1500 to 2500 dynes at the time point indicated in dotted lines, to thereby reduce RVAD preload. As shown, the compliant cannula is able to counteract the reduced RVAD preload, thereby maintaining flow, pressure and volume as compared to the rigid cannula.

In FIGS. 4E and 4F, left atrial cannulation (LAP) and left ventricular cannulation (Systolic LVvol) respectively, are shown for an LVAD with a progressively increasing impeller speed. In FIGS. 4G and 4H, right atrial cannulation (RAP) and right ventricular cannulation (Systolic RVvol) respectively, are shown for an RVAD with a progressively increasing impeller speed. Again, these results highlight the ability of the compliant inlet cannula to accommodate changes in blood flow to the heart pump as contrasted to the use of a non-compliant (semi-rigid) cannula.

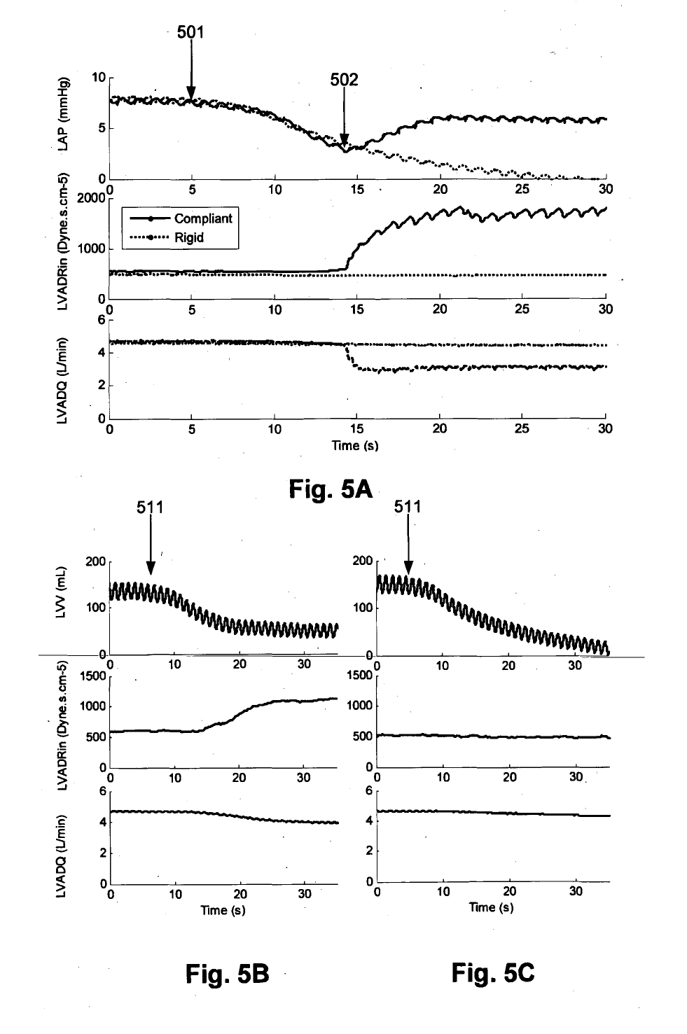

Further examples will now be described with reference to FIGS. 5A to 5C.

In FIG. 5A, left atrial cannulation is shown for an LVAD, with the solid and dotted lines representing a compliant and rigid inflow cannula respectively. In this example, the pulmonary vascular resistance (PVR) is increased from 100 to 400 dynes at the time point indicated by the arrow 501, to thereby reduce LVAD preload. As shown, the left atrial pressure (LAP) initially decreases, so that the inlet pressure decreases. When this decreases sufficiently, as shown by the arrow 502, the compliant inlet cannula restricts, thereby increasing the inlet resistance (LVADRin) and reducing the inflow rate (LVADQ). Consequently, the left atrial pressure returns to a higher level, whereas this correction is not achieved when the rigid cannula is used. Accordingly, the compliant cannula is able to counteract the reduced LVAD preload, thereby maintaining flow, pressure and volume as compared to the rigid cannula.

In FIGS. 5B and 5C, the effects of compliant and rigid inlet cannulae on left ventricular volume are shown for an LVAD. In this example, the pulmonary vascular resistance (PVR) is increased from 100 to 400 dynes at the time point indicated by the arrow 511, to thereby reduce LVAD preload. As shown in FIG. 5B, the compliant cannula is able to counteract the reduced LVAD preload by contracting and increasing the inflow resistance, thereby maintaining flow, pressure and volume as compared to the rigid cannula shown in FIG. 5C.

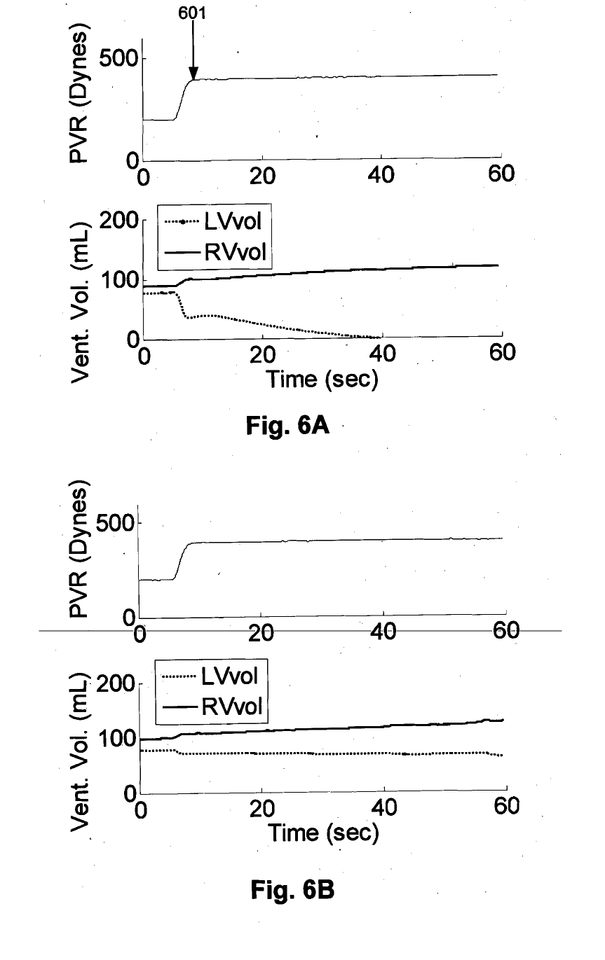

In FIGS. 6A and 6B, the effect of rigid and compliant outlet cannulae for an RVAD are shown. In this example, the pulmonary vascular resistance (PVR) is increased from 200 to 400 dynes·s·cm⁻⁵ at the time point indicated by the arrow 601, to thereby increase RVAD afterload and reduce LVAD preload.

As shown in FIG. 6B, the compliant outflow cannula is able to expand and counteract the increased RVAD preload, thereby maintaining flow, pressure and volume as compared to the rigid outflow cannula shown in FIG. 6A. Measured flows for an example heart pump including a rigid outflow cannula and compliant outflow cannula are shown in Tables 1 and 2, respectively.

TABLE 1
RIGID OUTFLOW

| | | | | | |
|---|---|---|---|---|---|
| PVR (Dynes) | 140 | 240 | 340 | 440 | 540 |
| LAP (mmHg) | 5.5 | 4.1 | 3 | 1.6 | 0.6 |
| MPQ (L/min) | 4.9 | 4.75 | 4.65 | 4.6 | 4.5 |
| MSQ (L/min) | 5.2 | 5.14 | 5.06 | 5 | 4.5 |
| MAP (mmHg) | 100 | 98 | 98 | 97 | 96 |
| MPAP (mmHg) | 15 | 18 | 23 | 27 | 31 |
| LV (mL) | 110 | 77 | 42 | 10 | −20 |
| RV (mL) | 90 | 98 | 108 | 116 | 125 |
| RVADRout | 2070 | 2050 | 2080 | 2050 | 2020 |

where: PVR—pulmonary vascular resistance
LAP—left atrial pressure
MPQ—mean pulmonary flow rate
MSQ—mean systemic flow rate
MAP—mean aortic pressure
MPAP—mean pulmonary arterial pressure
LV—left ventricle volume
RV—right ventricle volume
RVADRout—right ventricular assist device outflow cannula resistance

TABLE 2
COMPLIANT OUTFLOW

| | | | | | | | |
|---|---|---|---|---|---|---|---|
| PVR (Dynes) | 140 | 240 | 340 | 440 | 540 | 640 | 740 |
| LAP (mmHg) | 5.5 | 5.4 | 4.8 | 4.2 | 3.5 | 2.5 | 1.8 |
| MPQ (L/min) | 4.9 | 4.85 | 4.82 | 4.78 | 4.75 | 4.70 | 4.68 |
| MSQ (L/min) | 5.25 | 5.25 | 5.24 | 5.20 | 5.18 | 5.13 | 5.10 |
| MAP (mmHg) | 100.6 | 100 | 100.2 | 99.8 | 99.2 | 98.8 | 98.4 |
| MPAP (mmHg) | 14.1 | 20 | 25.3 | 30.5 | 35.5 | 40.5 | 45 |
| LV (mL) | 107 | 103 | 83.1 | 66.5 | 45 | 23.4 | 0 |
| RV (mL) | 86 | 83 | 86.5 | 88.2 | 92 | 95.8 | 99.9 |
| RVADRout | 1550 | 1460 | 1400 | 1340 | 1280 | 1220 | 1140 |

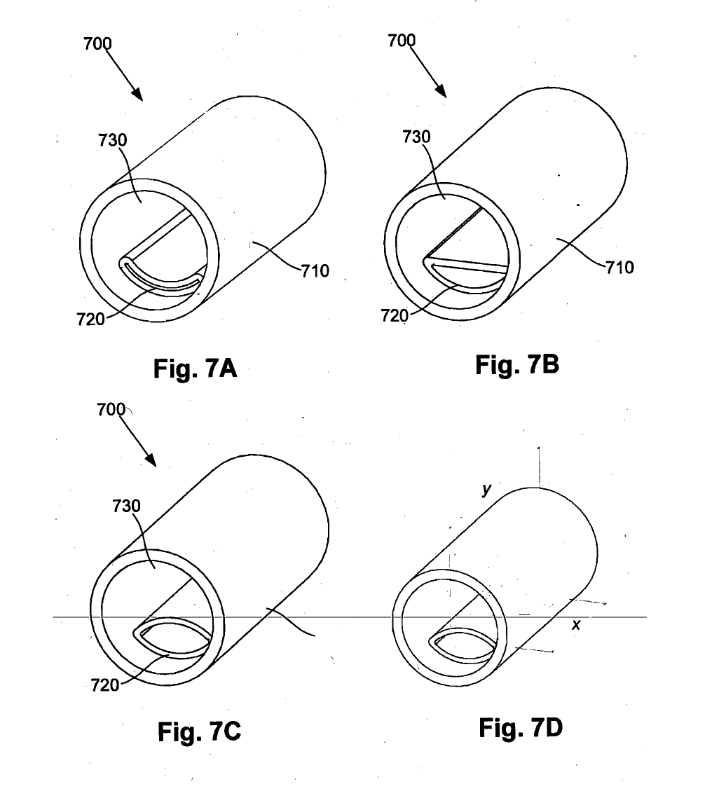

A further example of a compliant section will now be described with reference to FIGS. 7A to 7D.

In this example, the compliant section 700 includes an outer rigid casing 710 surrounding an inner tube 720. The inner tube is either deformable or only weakly compliant, for example if it is made of a woven fabric, such as a graft tube material including Dacron, or it is in the form of a thin silicone walled cannula. A compressible fluid, such as an inert gas is provided in a region 730 between the inner tube and the casing, to thereby provide additional compliance. In this example, the outer rigid casing and tube are gas impermeable, for example by manufacturing these from a gas impermeable material, or providing gas impermeable coatings.

In the above example, the total compliance is the combination of any tube compliance and compliance provided by the contained gas, which in turn will depend on the volume and pressure of the gas. When the outer casing has a length y, and the internal tube length x, shown in FIG. 7D, the ratio of the tube length to the outer rigid casing length determines compliance caused by the surrounding gas. In the event that the outer casing is longer than the inner tube, the difference is made up by a rigid, non-compliant inner tube of length y-x.

In the example of FIG. 7A, at a low internal blood pressure the tube 720 is in a restricted configuration, in which the tube is collapsed and has a zero or low starting cross-sectional area. In this arrangement, the initial internal gas pressure determines the required internal blood pressure for the tube to begin expanding, with this pressure typically being about atmospheric pressure 0 mmHg, or just below atmospheric, such as −3 mmHg.

As shown in FIG. 7B, in this example an internal blood pressure of about 20 mmHg expands the tube to a half open state, corresponding to a cross sectional area of a 5 mm diameter tube, whilst a further rise in internal blood pressure to about 70 mmHg expands the tube to a fully open state, corresponding to a cross sectional area of a 7 mm diameter tube.

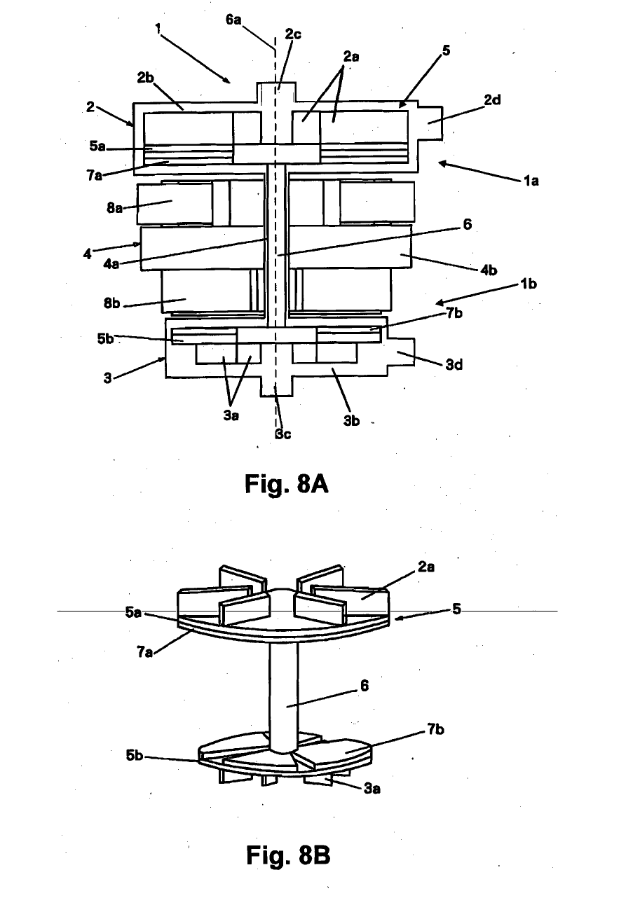

As apparent from the above described examples, the compliant section can be used with a wide range of heart pumps. A third example of a heart pump will now be described with reference to FIGS. 8A and 8B.

In this example, a first pump 2 is arranged in a first part 1a of the pump apparatus 1, and a second pump 3 is arranged in a second part 1b of the pump apparatus 1. In this example, at least part of a magnetic bearing and/or a drive, in this example a stator 4, is provided between the first and the second pumps 2, 3.

The pump apparatus includes an impeller 5, having a first rotor 5a and a second rotor 5b arranged in the first and second parts 1a, 1b, with the stator 4 being provided there between. The first and second rotors 5a, 5b are connected via a shaft 6 rotatably mounted within the pump apparatus to allow rotation about, and axial movement along an axis 6a in the connecting tube 4a, which in this example extends through the stator 4.

Thus, it will be appreciated that in this example, the connecting tube 4a is provided as a centre-hole through the stator 4, so that the stator 4 is provided radially outwardly of the connecting tube 4a. However, this is not essential and other arrangements can be used. For example, the connecting tube could be annular in shape, with the shaft being a hollow cylinder or a plurality of rods extending through the annular connecting tube.

The first rotor 5a is disk-shaped, and supports a first impeller 2a comprising a plurality of vanes that are provided on a first surface thereof. A first magnetic material in the form of a permanent magnet 7a is provided on a second surface of the first rotor 5a, facing the stator 4. The second rotor 5b is also disk-shaped, and supports a second impeller 3a composed of a plurality of vanes that are provided on a first surface thereof, with a second magnetic material, in the form of a plurality of permanent magnets 7b radially arranged on a second surface thereof, facing the stator 4. The permanent magnets 7b typically include a number of circumferentially spaced permanent magnets mounted in the impeller 5, adjacent magnets having opposing polarities, however, other suitable arrangements may be used, such as a Halbach array.

The stator 4 is composed of a doughnut-shaped body 4b, and first electromagnetic means 8a composed of, for example, four electromagnets, provided on a first surface of the body 4b, facing the first rotor 5a. The first electromagnetic means 8a generates a magnetic field that cooperates with permanent magnet 7a, allowing the axial position of the impeller 5 to be controlled.

Thus, the first electromagnetic means 8a and the first magnetic material form a magnetic bearing including at least one bearing coil for controlling an axial position of the impeller 5. In one example, the at least one bearing coil generates a magnetic field that is one of complementary to and counter to the magnetic field generated by the permanent magnet 7a, thereby allowing the impeller 5 to be levitated.

In addition, second electromagnetic means 8b is provided on a second surface of the body 4b facing the second rotor 5b that is, for example, composed of twelve three-phase electromagnets for generating a rotating magnetic field, whereby the electromagnets and the permanent magnet 7b cooperate with each other to thereby rotationally drive the second rotor 5b.

Accordingly, the second electromagnetic means 8b and magnets 7b form a drive for rotating the impeller. The drive therefore typically includes a second magnetic material provided in the impeller and at least one drive coil that in use generates a magnetic field that cooperates with the second magnetic material allowing the impeller to be rotated.

The first pump 2 includes a first pump chamber (or cavity portion) 2b and the first impeller 2a. The first impeller 2a can move axially while rotating with the first rotor 5a in the first pump chamber 2b. A first inlet port 2c is provided in a centre of an outer surface wall of the first pump chamber 2b, with an outlet port 2d being provided on a side wall of the first pump chamber 2b.

The second pump 3 includes a second pump chamber 3b and the second impeller 3a. The second impeller 3a can move axially while rotating with the second rotor 5b in the second pump chamber 3b. A second inlet port 3c is provided in a centre of an outer surface wall of the second pump chamber 3b, with an outlet port 3d being provided on a side wall of the second pump chamber 3b.

It will be appreciated that the pump chambers 2b, 3b may be provided with volutes to thereby assist with transfer of the fluid to the outlets 2d, 3d. The volutes maybe any combination of type spiral/single, split/double or circular/concentric, however the latter circular volute type is preferred if a journal bearing is used, as will be described in more detail below, as this configuration produces a stabilising radial hydraulic force for optimal journal bearing functionality.

It will therefore be appreciated that compliant sections associated with the inlet and/or outlet can be used to provide a passive flow modulation mechanism for left and/or right ventricular and/or biventricular assist device (VAD) and/or total artificial heart (TAH). The above described compliant section arrangement can also be used with a wide range of different rotary heart pumps, and their explanation of use with a magnetically levitated impeller is therefore for the purpose of illustration only.

Whilst the above examples have focussed on the use of the compliant section with an inlet and outlet of a heart pump, it will be appreciated that the same techniques could be applied to cannula as well as more generally to any apparatus for transporting fluid within a biological subject. In particular, the use of a compliant section allows the cannula or other apparatus to respond to changes in fluid pressure to thereby control the flow of fluid therethrough.

It will be appreciated that the term cannula is intended to encompass any tube that can be inserted into a biological subject to allow fluid flow therethrough, for example to allow for the delivery or removal of fluid from the subject, or more typically to transport blood between blood vessels of the subject or to transfer blood to or from a heart pump or other blood flow device.

The term biological subject is intended to encompass human subjects, but it will be appreciated that the techniques described above can be used with any animal, including but not limited to, primates, livestock, performance animals, such race horses, or the like.

It will be appreciated that the term compliance is intended to refer to a cannula section that is sufficiently compliant so that it will undergo a change in cross sectional area in response to hemodynamic pressure changes within the cannula, thereby allowing the flow of blood through the cannula to be modified, which in turn can be used to counteract the change in pressure.

Persons skilled in the art will appreciate that numerous variations and modifications will become apparent. All such variations and modifications which become apparent to persons skilled in the art, should be considered to fall within the spirit and scope that the invention broadly appearing before described.

The claims defining the invention are as follows:

1) Apparatus for transporting fluid within a biological subject, the apparatus including an at least partially compliant section, so that the compliant section at least partially controls the flow of fluid through the apparatus.

2) Apparatus according to claim 1, wherein a cross sectional area of at least part of the compliant section is dependent at least partially on the pressure of fluid therein, the cross sectional area of the compliant section controlling the flow of fluid therethrough.

3) Apparatus according to claim 2, wherein the cross sectional area of at least part of the compliant section is dependent on a pressure gradient across a wall of the compliant section.

4) Apparatus according to any one of the claims 1 to 3, wherein compliance is provided by at least one of:
a) a resilient band;
b) a compressible fluid; and,
c) material properties of the compliant section.

5) Apparatus according to any one of the claims 1 to 4, wherein the compliant section includes a deformable tube.

6) Apparatus according to claim 5, wherein the compliant section includes a resilient band provided around at least part of the deformable tube.

7) Apparatus according to any one of the claims 1 to 6, wherein the compliant section at least one of:
a) includes a rigid external casing;
b) is mounted within a rigid external casing;
c) includes a rigid external support;
d) includes a semi-rigid external wire reinforcement.

8) Apparatus according to any one of the claims 1 to 7, wherein the compliant section includes:
a) a substantially rigid outer casing;
b) a deformable tube provided within the casing; and,
c) a compressible fluid provided between the deformable tube and the casing.

9) Apparatus according to any one of the claims 1 to 8, wherein at least part of the compliant section is made of at least one of:
a) silicone;
b) a woven fibre material; and,
c) silastomer P15.

10) Apparatus according to any one of the claims 1 to 9, wherein in an expanded state at least part of the compliant section has an inner diameter of at least one of:
a) between 1 mm and 25 mm;
b) between 5 mm and 15 mm; and,
c) about 12 mm.

11) Apparatus according to any one of the claims 1 to 10, wherein in a restricted state at least part of the compliant section has an inner diameter of at least one of:
a) between 1 mm and 10 min;
b) between 2 mm and 8 mm; and,
c) about 6 mm.

12) Apparatus according to any one of the claims 1 to 11, wherein the compliant section has a wall thickness of at least one of:
a) between 0.5 mm and 3 mm; and,
b) about 1 mm.

13) Apparatus according to any one of the claims 1 to 12, wherein the compliant section has a compliance of at least one of:
a) between 0.2 mL/mmHg and 0.01 mL/mmHg;
b) between 0.1 mL/mmHg and 0.05 mL/mmHg;
c) about 0.08 mL/mmHg;
d) between 0.02 mL/mmHg and 0.001 mL/mmHg;
e) between 0.01 mL/mmHg and 0.005 mL/mmHg; and,
f) about 0.007 mL/mmHg.

14) Apparatus according to any one of the claims 1 to 13, wherein the apparatus includes a cannula.

15) Apparatus according to claim 14, wherein the cannula is attached to at least one of an inlet and an outlet of a heart pump.

16) Apparatus according to any one of the claims 1 to 15, wherein the apparatus includes a heart pump, the compliant section being associated with at least one of an inlet and an outlet of the heart pump.

17) Apparatus according to claim 15 or claim 16, wherein at least part of an inlet compliant section moves between an expanded state under conditions of normal inlet pressure and a restricted state under conditions of reduced inlet pressure, thereby reducing blood flow through the heart pump as the inlet pressure decreases.

18) Apparatus according to any one of the claims 15 to 17, wherein at least part of an outlet compliant section moves between a restricted state under conditions of normal outlet pressure and an expanded state under conditions of increased outlet pressure, thereby increasing blood flow through the pump as the outlet pressure increases.

19) A heart pump for pumping blood, the heart pump including:
a) a cavity including at least one inlet and at least one outlet;
b) an impeller provided within the cavity, the impeller including vanes for urging blood from the inlet to the outlet; and,
c) a drive for rotating the impeller in the cavity; and,
d) an at least partially compliant section associated with at least one of the inlet and the outlet, wherein in use the compliant section at least partially controls the flow of blood through the heart pump.

20) A heart pump according to claim 19, wherein a cross sectional area of at least part of the compliant section is at least partially dependent on the pressure of blood therein, the cross sectional area of the compliant section controlling the flow of blood through the heart pump.

21) A heart pump according to claim 20, wherein the cross sectional area of at least part of the compliant section is dependent on a pressure gradient across a wall of the compliant section.

22) A heart pump according to any one of the claims 19 to 21, wherein at least part of an inlet compliant section moves between an expanded state under conditions of normal inlet pressure and a restricted state under conditions of reduced inlet pressure, thereby reducing blood flow through the pump as the inlet pressure decreases.

23) A heart pump according to any one of the claims 19 to 22, wherein at least part of an outlet compliant section moves between a restricted state under conditions of normal outlet pressure and an expanded state under conditions of increased outlet pressure, thereby increasing blood flow through the pump as the outlet pressure increases.

24) A heart pump according to any one of the claims 19 to 23, wherein the compliant section is part of at least one of:
a) the inlet;
b) the outlet;
c) a volute associated with the outlet;
d) an inlet cannula coupled to the inlet for receiving blood from a subject; and,
e) an outlet cannula coupled to the outlet for supplying blood to the subject,

25) A heart pump according to any one of the claims 19 to 24, wherein compliance is provided by at least one of:
a) a resilient band;
b) a compressible fluid; and,
c) material properties of the compliant section.

26) A heart pump according to any one of the claims 19 to 25, wherein the compliant section includes a deformable tube.

27) A heart pump according to claim 26, wherein the compliant section includes a resilient band provided around at least part of the deformable tube.

28) A heart pump according to any one of the claims 19 to 27, wherein the compliant section at least one of:
a) a rigid external casing;
b) is mounted within a rigid external casing;
c) a rigid external support;
d) includes a semi-rigid external wire reinforcement.

29) A heart pump according to any one of the claims 19 to 28, wherein the compliant section includes:
a) a substantially rigid outer casing;
b) a deformable tube provided within the casing; and,
c) a compressible fluid provided between the deformable tube and the casing.

30) A heart pump according to any one of the claims 19 to 29, wherein at least part of the compliant section is made of at least one of:
a) silicone;
b) a woven fibre material; and,
c) silastomer P15.

31) A heart pump according to any one of the claims 19 to 28, wherein in an expanded state at least part of the compliant section has an inner diameter of at least one of:
a) between 1 mm and 25 mm;
b) between 5 mm and 15 mm; and,
c) about 12 mm.

32) A heart pump according to any one of the claims 19 to 31, wherein in a restricted state at least part of the compliant section has an inner diameter of at least one of:
a) between 19 mm and 190 mm;
b) between 2 mm and 8 mm; and,
c) about 6 mm.

33) A heart pump according to any one of the claims 19 to 32, wherein the compliant section has a wall thickness of at least one of:
a) between 0.5 mm and 3 mm; and,
b) about 1 mm.

34) A heart pump according to any one of the claims 19 to 33, wherein the compliant section has a compliance of at least one of:
a) between 0.2 mL/mmHg and 0.01 mL/mmHg;
b) between 0.1 mL/mmHg and 0.05 mL/mmHg;
c) about 0.08 mL/mmHg;
d) between 0.02 mL/mmHg and 0.001 mL/mmHg;
e) between 0.01 mL/mmHg and 0.005 mL/mmHg; and,
f) about 0.007 mL/mmHg.

35) A heart pump according to any one of the claims 19 to 34, wherein the drive includes:
a) a first magnetic material provided in the impeller;
b) at least one drive coil that in use generates a magnetic field that cooperates with the first magnetic material allowing the impeller to be rotated.

36) A heart pump according to claim 35, wherein the first magnetic material includes a number of circumferentially spaced permanent magnets mounted in the impeller, adjacent magnets having opposing polarities.

37) A heart pump according to any one of the claims 19 to 36, wherein the pump includes a magnetic bearing including at least one bearing coil for maintaining an axial position of the impeller.

38) A heart pump according to claim 37, wherein, in use, the at least one bearing coil generates a magnetic field that cooperates with second magnetic material in the impeller, allowing the axial position of the impeller to be maintained.

39) A heart pump according to claim 38, wherein the second magnetic material is a permanent magnet.

40) A heart pump according to claim 39, wherein the at least one bearing coil is for generating a magnetic field that is one of complementary to and counter to the first magnetic field generated by the permanent magnet, thereby controlling the net magnetic field between the bearing and the first magnetic material.

41) A heart pump according to any one of the claims 19 to 40, wherein the heart pump includes:
a) a first cavity portion having a first inlet and a first outlet;
b) a second cavity portion having a second inlet and a second outlet; and,
c) first and second sets of vanes provided on the impeller, each set of vanes being for urging fluid from a respective inlet to a respective outlet.

42) A heart pump according to claim 41, wherein the pump includes a connecting tube extending between the first and second cavity portions, the impeller including:
a) a first set of vanes mounted on a first rotor in the first cavity portions;
b) a second set of vanes mounted on a second rotor in the second cavity portion; and,
c) a shaft connecting the first and second rotors, the shaft extending through the connecting tube.

43) A pump according to claim 42, wherein the at least one of the drive and a magnetic bearing are provided at least partially between the first and second cavity portions

44) A heart pump according to any one of the claims 38 to 43, wherein first magnetic material is mounted in a first rotor supporting the first set of vanes and wherein the magnetic bearing is positioned adjacent the first cavity, the magnetic bearing and first magnetic material being configured to result in an attractive force between the magnetic bearing and the first rotor.

45) A heart pump according to any one of the claims 38 to 44, wherein second magnetic material is mounted in a second rotor supporting the second set of vanes and wherein the magnetic bearing is positioned adjacent the second cavity, the magnetic bearing and second magnetic material being configured to result in an attractive force between the magnetic bearing and the second rotor.

46) A cannula for transporting fluid within a biological subject, the cannula including an at least partially compliant section, so that the compliant section at least partially controls the flow of fluid through the cannula.

47) A cannula according to claim 46, wherein a cross sectional area of at least part of the compliant section is dependent at least partially on the pressure of fluid therein, the cross sectional area of the compliant section controlling the flow of fluid therethrough.

48) A cannula according to claim 47, wherein the cross sectional area of at least part of the compliant section is dependent on a pressure gradient across a wall of the compliant section.

49) A cannula according to any one of the claims 46 to 48, wherein compliance is provided by at least one of:
a) a resilient band;
b) a compressible fluid; and,
c) material properties of the compliant section.

50) A cannula according to any one of the claims 46 to 49, wherein the compliant section includes a deformable tube.

51) A cannula according to claim 50, wherein the compliant section includes a resilient band provided around at least part of the deformable tube.

52) A cannula according to any one of the claims 46 to 51, wherein the compliant section at least one of:
a) includes a rigid external casing;
b) is mounted within a rigid external casing;
c) includes a rigid external support;
d) includes a semi-rigid external wire reinforcement.

53) A cannula according to any one of the claims 46 to 52, wherein the compliant section includes:
a) a substantially rigid outer casing;
b) a deformable tube provided within the casing; and,
c) a compressible fluid provided between the deformable tube and the casing.

54) A cannula according to any one of the claims 46 to 53, wherein at least part of the compliant section is made of at least one of:
a) silicone;
b) a woven fibre material; and,
c) silastomer P15.

55) A cannula according to any one of the claims 46 to 54, wherein in an expanded state at least part of the compliant section has an inner diameter of at least one of:
a) between 1 mm and 25 mm;
b) between 5 mm and 15 mm; and,
c) about 12 mm.

56) A cannula according to any one of the claims 46 to 55, wherein in a restricted state at least part of the compliant section has an inner diameter of at least one of:
a) between 1 mm and 10 mm;
b) between 2 mm and 8 mm; and,
c) about 6 mm.

57) A cannula according to any one of the claims 46 to 56, wherein the compliant section has a wall thickness of at least one of:
a) between 0.5 mm and 3 mm; and,
b) about 1 mm.

58) A cannula according to any one of the claims 46 to 57, wherein the compliant section has a compliance of at least one of:
a) between 0.2 mL/mmHg and 0.01 mL/mmHg;
b) between 0.1 mL/mmHg and 0.05 mL/mmHg;
c) about 0.08 mL/mmHg;
d) between 0.02 mL/mmHg and 0.001 mL/mmHg;
e) between 0.01 mL/mmHg and 0.005 mL/mmHg; and,
f) about 0.007 mL/mmHg.
